(19) United States
(12) Patent Application Publication
(10) Pub. No.: US 2021/0187142 A1
(43) Pub. Date: Jun. 24, 2021

(54) DISINFECTION SYSTEM

(71) Applicant: UAB "AIRPLUS 1 LITUANICA", Klaipeda (LT)

(72) Inventor: Saulius CAJAUSKIS, Kunkiai (LT)

(21) Appl. No.: 16/076,254

(22) PCT Filed: Jan. 25, 2017
(86) PCT No.: PCT/IB2017/050374

§ 371 Date: Aug. 7, 2018

(30) Foreign Application Priority Data
Feb. 8, 2016 (GB) 1602191.7
Jul. 13, 2016 (LT) LT2016082
Jul. 29, 2016 (LT) LT2016086

(51) Int. Cl. A61L 2/20; A61L 11/00; A61L 2/24; A61L 9/015; B01D 53/46

(52) U.S. Cl. CPC A61L 2/202; A61L 11/00; A61L 2/24; A61L 9/015; B01D 2258/06; A61L 2202/14; A61L 2209/11; B01D 2251/104; B01D 2257/91; B01D 53/46

(57) ABSTRACT

A disinfection system includes at least one ozone generator; at least one ozone concentration level detector; programmable timer; at least one presence detector; at least one main system process management tool; at least one intermediate system process management tool. This invention aims not only to disinfect the air by removing unpleasant odour, but also to disinfect wastewater, premises and facilities by adjusting ozone concentration. Thus, not only the consequence of bad odour, which appears due to bacteria, protozoa, moulds and other fungi, but also the cause for occurrence of such bad odour is eliminated by exterminating viruses, bacteria, fungi, mould and all the main biogases within the disinfected environment. Modifications of this invention allow adjusting ozone concentration in order to disinfect premises and environment by avoiding possible hazardous ozone impact on people, working in the environments to be disinfected or residing near to such environments.

### TECHNICAL AREA

This invention is related to a disinfection system and, particularly, to a system and equipment for disinfecting various media with ozone.

### TECHNICAL LEVEL

As a result of growth and expansion of cities and settlements globally, society generates significant amounts of domestic and industrial wastewater.

Issues of disinfecting wastewater pumping stations and wastewater treatment plants (industrial and private) and neutralising bad odour are causing increasingly more concern. Residents are complaining about worsening health, constant headaches, nausea, increase in cases of depression and sudden mood swings. And this is completely understandable as well as approved by research studies [Assoc. Prof. Dr. Dainius Paliulis and Dr. Eglė Zuokaitė, Methodical Recommendations for Odour Management]. Wastewater pumping stations and industrial and public wastewater and its content within wastewater treatment plants become the medium for growth of microorganisms (particularly at higher temperatures), thus resulting in additional pollutants, such as bacteria, protozoa, moulds, etc., which in turn produce other organic pollutants. Fungi, bacteria and gases not only accumulate in wastewater pumping stations and wastewater treatment plants, but are also transmitted through the air, particularly in the areas where residential and civil buildings are in close proximity to wastewater pumping stations. For this reason, significant risk is posed to human and animal health. When disinfection is carried out by using water and chemicals, disinfection mixture does not always penetrate all the crevices and cracks of the wastewater collection and recycling system that may contain sources of bad sources and contamination.

Another source of bad odour and contamination is depositories of public waste in residential buildings, where significant amount of building's waste is accumulated. Such waste, stored in repositories, usually include non-food, inorganic, food and other organic waste. Part of such waste and their content rot and become a medium for incubation of pests and also includes undesirable pollutants, such as bacteria, protozoa, fungi, moulds and other organic pollutants. In addition, fungi and bacteria not only accumulate in repository, but also can be transmitted through the air. Therefore, waste repositories pose risk to health, a poorly supervised waste repository can result in occurrence of pest transmitting infection and start spreading unpleasant odour. Similarly, the aforementioned issues often occur in buildings where waste collection chutes or pipes for throwing litter from other building floors to the waste repository are installed.

Similar issue is encountered in any premises or facilities, where certain conditions can cause fungus and/or bacteria that may pose risk to human health by spreading in the air. Or which result in at least unwanted/unpleasant odour. Such premises, premises/facilities may comprise premises that include air flow vent for ventilation, such as:

1) Premises for storage of food and noon-food materials.

2) Used container collection premises equipped with reverse vending machines.

3) Refrigerators (industrial and private).

4) Freezer storage facilities in seaports and airports.

5) Premises for storage of production, raw and finished products of food industry (meat, fish, milk, etc.).

6) Facilities of healthcare institutions that require disinfection.

7) Hotel rooms or other premises (especially following the visits of customers who smoke).

8) Rooms for smoking.

9) Public catering facilities.

10) Public and private WCs.

11) Premises and facilities (industrial and private) for collection or pumping of domestic and industrial wastewater, ventilation systems and wastewater recycling localisation sites.

12) Ventilation systems for residential, industrial buildings and buildings of other purposes.

13) Public and freight transport.

14) Commercial premises.

15) Changing-rooms in gyms or swimming pools.

Disinfection systems are available for automated wastewater pumping stations and wastewater treatment plants (industrial and public) that neutralise and dissolve moulds, bacteria and other hazardous microorganisms, as well as biogas present both on surfaces and in the air. One of such systems is described in the patent description No. LT5977B (patent number 5977, publication date: 27 Dec. 2013), also described in the patent application No: 017228 (application No: 2016110902, the Russian Federation, date: 24 Mar. 2016), which particularly focuses on preventing wastewater odour from reaching residential houses. Based on the patent applications No. LT5977B (patent number 5977, publication date: 27 Dec. 2013) and No: 017228 (application No: 2016110902, the Russian Federation, date: 24 Mar. 2016) solution to the issue is approached in a superficial manner only, i.e. by dealing with the consequence of bad odours rather than the cause. Based on the patent application No: 017228, the problem in the wastewater pumping station is approached by installing methane sensor and, once increase in methane concentration is detected, by activating ozone generator in the wastewater pumping station. Ozone gas is an effective disinfectant that is capable of efficient disinfection of large volume of air in order to prevent accumulation of biofilms, bacteria, fungi and biogas in the wastewater pumping station. Using ozone generator in the wastewater pumping station is an efficient way to neutralise unpleasant odours and reduce incidence rate of health problems, such as allergies, rashes, cold and legionellosis. It is also known that ozone prevents distribution of moulds and parasites as well as destroys large part of biogas. Hence, the system described in the patent description No. LT5977B and No: 017228 can significantly improve sanitary conditions.

Despite this, conventional wastewater odour removal system, described in the patent description No. LT5977B, has its disadvantages. Firstly, it generates ozone only when increased methane concentration is captured in the wastewater pumping station. When this system is used, the problem related to other pollutants present in the wastewater pumping station is not tackled directly. For instance, there may be periods when wastewater fermentation and decomposition processes take longer time due to lower air temperature. In such cases, quantity of released methane decreases respectively, therefore the system becomes less efficient when tackling the problem of residual biofilms, moulds and other pests as well as biogases. In addition, although the system is an efficient measure to prevent wastewater odour from spreading, instead, ozone gas odour may occur that is itself sufficiently pungent and toxic. Ozone gas is also a hazardous substance and its high concentration in the wastewater pumping station may be unpleasant for people residing nearby. Sustained high levels of ozone exposure can also lead to headaches, eye and respiratory irritation and even longer-lasting negative consequences, including lung damage. The system description does not contain suggestions for modifications in order to adjust quantity of produced ozone, covering use of ozone sensors for monitoring ozone level in the wastewater pumping station and deactivating ozone generator when concentration exceeds the set threshold. Although such simplified safety deactivation mechanism can prevent from occurrence of hazardous significant ozone level, other factors that may affect identification of acceptable ozone concentration level have not been taken into account.

There are automated disinfection systems for waste container repositories available that neutralise and dissolve moulds, bacteria and other hazardous microorganisms, as well as biogas present both on surfaces and in the air. One of such systems is described in the patent application No. U.S. Ser. No. 14/532,867 (publication number US 2015/0157753), which particularly focuses on preventing waste odour from reaching residential floors of buildings through the chute holes present in each floor of residential buildings. Based on the patent application No. U.S. Ser. No. 14/532,867, top part of the chute is equipped with methane sensor and, once there is an increase in methane concentration, ozone generator is activated in the waste depository. Ozone gas is an effective disinfectant that is capable of efficient disinfection of large volume of air in order to prevent accumulation of biofilms, bacteria, and fungi in the waste repository and connected chutes. Using ozone generator in the waste repository is an efficient way to neutralise unpleasant odours and reduce incidence rate of health problems, such as allergies, rashes, cold, virus diseases, and legionellosis. It is also known that ozone prevents distribution of moulds and parasites. Hence, the system described in the patent application No. U.S. Ser. No. 14/532,867 can significantly improve sanitary conditions.

Despite this, conventional waste odour removal system, described in the patent application No. U.S. Ser. No. 14/532,867, has its disadvantages. Firstly, based on the patent application No. U.S. Ser. No. 14/532,867, the system generates ozone only when increased methane concentration is captured at the top of the waste chute. Based on the patent application No U.S. Ser. No. 14/532,867, the system is suitable only for collecting waste within the systems with waste chutes. When the system is used according to patent application No. U.S. Ser. No. 14/532,867, the problem of other pollutants that can be present in the very same waste repository, may not be tackled directly. For instance, there may be periods when wastewater fermentation and decomposition processes take longer time due to lower air temperature. In such cases quantity of released methane also decreases respectively, therefore, according to the patent application No. U.S. Ser. No. 14/532,867, the system becomes less efficient when tackling the problem of residual biofilms, moulds and other pests that can still be present in the waste repository.

Although the system described in the patent application No. U.S. Ser. No. 14/532,867 is an efficient measure to prevent spreading of waste odour from the waste chute holes, ozone gas odour may occur that is itself sufficiently pungent. Ozone gas is also a hazardous substance and its high concentration in the depository may be unpleasant for people residing nearby or for those who are frequently visiting the water repository with a purpose, e.g. to empty the waste containers. Sustained high levels of ozone exposure can also lead to headaches, eye and respiratory irritation and even longer-lasting negative consequences, including lung damage. Modifications are suggested in the description of the system for adjustment of the produced ozone quantity in accordance with the patent application No U.S. Ser. No. 14/532,867, covering use of ozone sensors for monitoring of ozone level in the waste chutes and repository as well as deactivation of ozone generator when concentration exceeds the set threshold. Although such simplified safety deactivation mechanism can prevent from occurrence of hazardous significant ozone level, other factors that may affect identification of acceptable ozone concentration level have not been taken into account.

This invention aims to solve some issues that occur while using conventional disinfection systems, intended for disinfection of various media by using ozone.

### BRIEF DESCRIPTION OF THE INVENTION

According to the first aspect of this invention, a disinfection system is provided for installation in wastewater pumping stations, comprising the following: ozone generators for generation of ozone intended for disinfection of air and the waste in the waste pumping stations; an ozone detector for measurement of ozone concentration of wastewater pumping stations; an airflow detector; an intermediate controller for collection and transfer of data and a central controller for management of the ozone generator so that ozone would be generated depending on the measured ozone concentration and set time of a day.

According to the second aspect of this invention, a disinfection system is provided for installation in the wastewater treatment plants (open wastewater storage tanks), covering ozone generators for generating ozone directly in wastewater, which are installed on an open side of a wastewater treatment tank intended for disinfection by directing the produced ozone directly to wastewater through ozone-resistant pipes; ozone detectors for measuring ozone concentration in the environment, mounted around the perimeter of the wastewater tank; an intermediate controller for collection and transfer of data and a central controller for management of the ozone generators, so that ozone would be generated depending on the measured ozone concentration.

According to the third aspect of this invention, a waste disinfection system is provided for installation in a waste depository, comprising: an ozone generator for generating ozone in the repository; an ozone detector for measuring ozone concentration in the waste chute pipe of the waste repository; an airflow detector; an intermediate controller for collection and transfer of data and a central controller for management of the ozone generator, so that ozone would be generated depending on the measured ozone concentration and set time of a day.

According to the fourth aspect of this invention, a facility disinfection equipment is provided, an comprising ozone generator for generating ozone in rooms, installed on a wall/ceiling of a room to be disinfected; an ozone detector for measuring ozone concentration in the room, installed separately from the ozone generator, on the opposite wall of the room to be disinfected; an ozone detector in a vent of the room; an airflow detector in the vent of the room; an intermediate controller for collection and transfer of data and a central controller for management of the ozone generator so that ozone would be generated depending on the measured ozone concentration and set time of a day.

This invention aims not only to disinfect air by removing an unpleasant odour, but also to directly disinfect wastewater, premises and facilities by adjusting ozone concentration. Thus, not only the consequence of bad odour, which appears due to bacteria, protozoa, moulds and other fungi, but also the cause for occurrence of such bad odour is eliminated by exterminating viruses, bacteria, fungi, mould and all the main biogases within the disinfected environment. Modifications of this invention allow adjusting ozone concentration in order to disinfect premises and environment by avoiding possible hazardous ozone impact on people, working in the environments to be disinfected or residing near to such environments. In addition, use of feedback systems guarantee that mandatory requirements for ozone concentration determined by respective regulatory authorities shall not be exceeded.

### BRIEF DESCRIPTION OF DRAWINGS

Other features and advantages of the invention are described in the details of the invention with a link to the following drawings:

FIG. 1 shows the system for disinfecting wastewater pumping stations with ozone, based on the first modification of this invention;

FIG. 2 shows the system for disinfecting wastewater treatment plants (open wastewater collection and treatment tanks) with ozone, based on the second modification of this invention.

FIG. 3 shows the system for disinfecting rooms with ozone, based on the third modification of this invention;

FIG. 4 shows the system for disinfecting rooms with ozone, based on the fourth modification of this invention.

Prior to providing detailed description of the invention with a link to drawings of the invention implementation examples, please note that identical elements are marked with the same numbers in all the drawings.

### DETAILED DESCRIPTION OF THE INVENTION

System intended for disinfecting at least one environment comprises the following: a vent; at least one ozone generator; a first ozone detector for measuring ozone concentration in proximity of at least one ozone generator; and a second ozone generator for measuring ozone concentration in the vent, in case of closed environment; a control relay in case of closed environment; an airflow detector in case of a closed environment; a timer; an intermediate controller; at least one other intermediate controller for collection and transfer of data and central controller for management of ozone generator, so that ozone would be generated depending on a measured ozone concentration and set time of a day; an occupation detector.

The system controls quantity of ozone in the disinfected environment twenty four hours a day in order to achieve optimal balance between efficient disinfection and safe user environment. For instance, in case of higher ozone concentration, disinfection is more efficient, however, maintaining such high ozone concentration during that time of a day when people can be affected, it poses significantly higher risk of unpleasant or hazardous ozone impact. This can be avoided, e.g., by maintaining higher concentration at a night time or any other time when no people are present in the environment that is to be disinfected, or near to it. This can increase ozone generation efficiency when dealing with microorganisms and pests and, at the same time, making sure that relevant legal thresholds are not exceeded.

Time of a day is determined by using a timer; its data is employed by the central controller to manage ozone generators. It is a simple way to control a 24-hour schedule, although other ways are also possible. For instance, outdoor light and/or temperature detectors can be installed in order to distinguish day and night time.

A central controller manages ozone generators according to the programmed schedule and set time of the day. When programming time schedule, one or more of the following components is taken into account: season, work days and holidays. In this way, quantity of ozone generated per day will vary depending on the time of the year or occupation model. For example, at lower temperatures during winter, wastewater odour is less of a problem, compared to the summer months.

The central controller stops producing ozone in the ozone generator once ozone detector identifies that ozone concentration exceeds the estimated stopping value in the disinfected environment. In this way, excess ozone production is avoided and, e.g., legal thresholds are not exceeded, above which people would be noticeably affected by ozone.

Concentration thresholds vary depending on the time of a day. When high ozone concentration is provided, threshold concentration is determined in accordance with legal thresholds, while during the time of the day when low ozone concentration is provided; a threshold is set so that people would not be affected by ozone.

The first ozone detector is installed at such a distance from ozone generators that ozone could mix with air prior to measuring concentration. This way ozone concentration is measured more precisely.

The disinfection system additionally includes a presence detector, intended for detection presence of people within the disinfected environment. Once the detector detects presence of a person within the disinfected environment, the central controller stops ozone generators. This serves as an additional safety measure: even though there would still be ozone remaining in the air, yet it wouldn't be actively generated and would dissipate. Thus, a person present in the disinfected environment could avoid long-term impact of high ozone concentration that could harm his/her health. Function of the presence detector can be performed by at least one motion detector. Various motion detectors can be used, such as infrared, microwave, ultrasound or tomography technologies. Other types of presence detectors also can be used, such as door sensors, which detect when doors in wastewater pumping stations are opened. In addition, to increase presence detection accuracy, combined sensors can be used in the detectors.

The disinfection system additionally comprises an airflow detector for measuring airflow coming from the closed disinfected environment, e.g., in a vent or an opening with a similar function, where then central controller stops ozone production in the ozone generators in case when airflow detector detects that airflow dropped below the threshold. Thus it can be quickly identified whether ventilation device of the disinfected environment system is clogged and thus prevent rapid accumulation of ozone gas.

The disinfection system additionally comprises the second ozone detector for measuring ozone concentration in a vent within closed environment, where central controller will stop ozone generators in case when the second ozone detector detects that the second threshold concentration is exceeded. This would be the second measure protecting against formation of excessive ozone concentration in the exhaust hole of the ventilation system.

Preferably, depending on the time of the day, different concentration values should be defined for the second threshold.

The disinfection system additionally comprises an intermediate controller, from where data collected from the system elements are transferred directly to the central controller. The central controller would allow remotely supervise disinfection system and control it. The central controller comprises input to obtain response data from several different disinfection systems through more than one intermediate controller. Such way would allow observing and comparing data from several wastewater pumping stations.

Response data can be transferred from the intermediate controller to the central controller via cable, through GSM module or internet connection.

### Examples

Understandably, many specific parts are laid out in order to provide full and comprehensive description of the invention implementation example. However, experts shall clearly see that specific details of the invention implementation examples do not limit the invention implementation, which can be carried out even without having such specific instructions. Well-known methods, procedures and components have not been described in detail, so that the invention implementation examples would not be misleading. Furthermore, this description should not be considered as limiting the provided implementation examples, but should be considered only as their implementation.

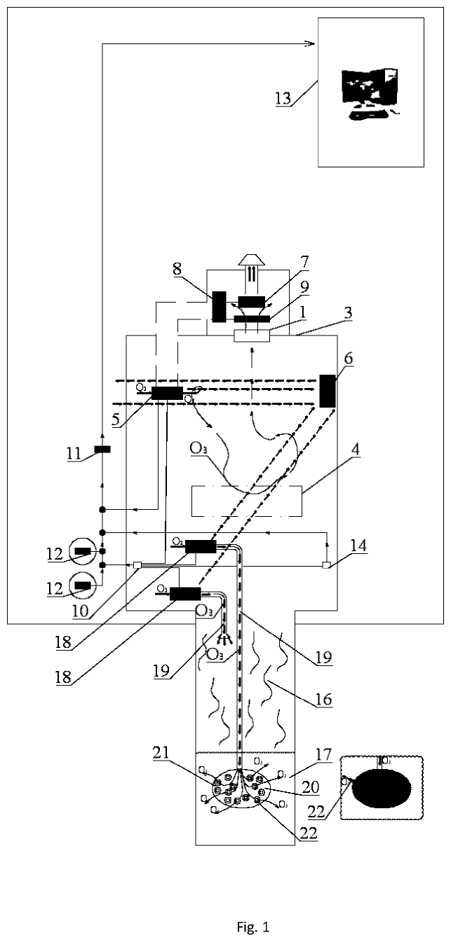

FIG. 1 shows the provided disinfection system intended for installing in premises of wastewater pumping stations (3). The system comprises at least three ozone generators (5, 18), where at least one ozone generator (18) is suitable for supplying ozone to the wastewater (17); the first ozone detector (6) intended for measuring ozone concentration in the wastewater room (3) and the second ozone detector (7) intended for measuring ozone concentration in the wastewater pumping stations, located in the vents of the system (1); an airflow detector (9); at least one intermediate controller (11) intended for collecting and transferring data to the central controller (13); at least one intermediate controller for other premises (12), intended for collecting and transferring data to the central controller (13) from other identical disinfection systems; a central controller (13) intended for managing ozone generators, so that it would generate ozone depending on the measured ozone concentration and set time of the day; a control relay (8); a timer (10); at least one presence detector (14); ozone-resistant ozone supply pipes (19); openings with reverse valves (21); reverse valves (22).

The disinfection system according to the first implementation modification includes ozone generators (5, 18), which spread ozone to the room to be disinfected (3), to wastewater collection shaft (16), as well as directly to the wastewater (17), and is managed through the central controller (13). Ozone release outlet of at least one ozone generator (5) is adapted for directing ozone gas flow to the room intended for the room to be disinfected and/or facility (4) to be disinfected within the room. Ozone produced by higher capacity generators (18) is released through ozone-resistant pipes (19), which can be connected to the ozone release head (20) that contains two holes (21) with reverse valve (22) to release ozone so that these holes would not be clogged with wastewater if ozone generators (18) stopped working.

In this modification, the central controller (13) is intended to receive control information through the intermediate controller (11) from the timer (10), the ozone detectors (6, 7) and the presence detector (14), as well as other identical intermediate controllers (12) intended for disinfection systems. The central controller (13) is intended for monitoring incoming control data and adjust settings in a centralised way by sending response instructions through the intermediate controller (11, 12) in order to manage the ozone generators (5, 18). The central controller (13) would allow to remotely monitor disinfection system in the wastewater pumping stations and control it. The central controller (13) includes input for receiving response data from more than one intermediate controller (11, 12), from multiple wastewater pumping stations. Such way would allow observing and comparing data from several wastewater pumping stations. Response data can be transferred from at least one intermediate controller (11, 12) to the central controller (13) by using a cable, a GSM module or an internet connection.

The room to be disinfected (3) contains the first ozone detector (6), intended for measuring ozone concentration inside the room (3); the presence detector (14) for detecting persons within the room (3) and the timer (10). All the detectors (6, 7, 14) transfer collected data to the central controller (13). The first ozone detector (6) is installed separately from the ozone generator (5); it is fitted to the wall in premises (3), away from the ozone generator, and preferably on the opposite wall in premises (3), to make both devices separated as much as possible. The aim is that generated ozone mixes up with room air before reaching the ozone detector (6). Thus, the first ozone detector (6) installed in the room measures ozone concentration more accurately.

When the first ozone detector (6) detects that ozone concentration in an environment exceeds the estimated stopping value, the central controller (13) stops the ozone generators (5, 18). In this way modifications would not generate excessive amounts of ozone. This possibility can be exploited, for example, in order to not exceed legal values and threshold above which people would be noticeably affected by ozone. Preferably, that different concentration limits would be set depending on the time of the day. Then, at the time of the day when high ozone concentration is provided, the threshold concentration can be determined in accordance with the legal thresholds, while at the time of the day when low ozone concentration is provided; a threshold could be set so that people would not be affected by the ozone. The first ozone detector (6) is installed at such a distance from ozone generators (5, 18) that ozone could mix with air prior to measuring concentration. This way ozone concentration is measured more precisely.

The presence detector (14) is installed on the wall of the room to be disinfected (3). The presence detector (14) sensor is directed to the place where only movements of a person entering the room to be disinfected (3) can be detected. Once a person is detected, the central controller (13) stops ozone generators (5, 18). This serves as an additional safety measure, since even though there would still be ozone remaining in the air within the room, yet it wouldn't be actively generated anymore and would dissipate. Thus a person could be safe in the room and avoid long-term impact of high ozone concentration that could impair his/her health. At least one motion detector can perform the function of presence detector (14). In this way persons present in the room could be detected based on their movement. Various motion detectors can be used, such as infrared, microwave, ultrasound or tomography technologies. Other types of presence detectors also can be used, such as door sensors, which detect when doors in wastewater pumping stations are opened. In addition, to increase presence detection accuracy, combined sensors can be used in the detectors.

The airflow detector (9) is installed at the top of the vent (1), by the junction with ventilation chimney and used for measuring of airflow released through the vent (1). Once airflow detector (9) detects that airflow falls below the threshold, the central controller (13) stops the ozone generators (5, 18). In this way it can be quickly determined that system ventilation device in the wastewater pumping stations is clogged and thus avoid rapid accumulation of ozone gases.

The system comprises an additional ozone detector (7), intended for measuring ozone concentration in the air released from the vent. It can be installed for measuring ozone level in the air released from the airflow detector (9). The control relay (8) is installed for transferring captured data to the intermediate controller (11, 12). Central controller stops the ozone generators (5, 18) when the second ozone sensor (7) detects that it exceeds the second concentration threshold value. This is the second safety measure protecting against formation of excessively high concentration of ozone penetrating through exhaust vent of the wastewater pumping station in the building's environment. Depending on the time of the day, it sets a different second threshold value. In this way the system operates more actively during that time of the day when vents in the wastewater pumping stations are less likely to be opened and thus higher ozone concentration would be created.

The timer (10) is intended for controlling time of the day and control activities based on the time schedule programme as well as to transfer data to the central controller (11, 12) in order to control ozone generators (5, 18) depending on the time of the day. Timer of this modification comprises a touch screen to enter the control options and to set the program time schedule. This is a simple way to control the timing of 24 hours according to the programmed schedule and set time of the day. When programming time schedule, one or more of the following components is taken into account: season, work days and holidays. Such way would allow for generated ozone vary within a day depending on a season and on a presence model. For example, at lower temperatures during winter, wastewater odour is less of a problem, compared to the summer months.

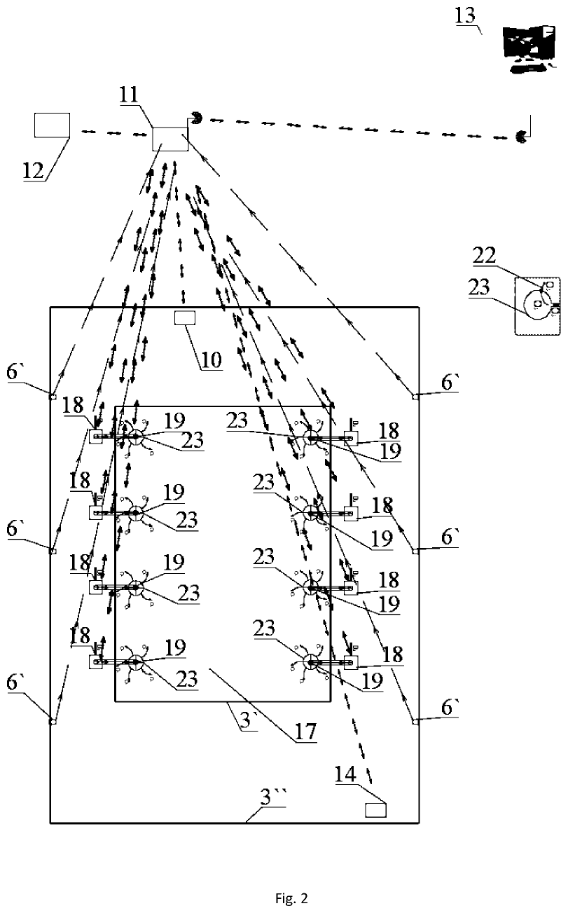

FIG. 2 shows the disinfection system for installation in wastewater treatment stations (open wastewater treatment tanks), where certain conditions may result in occurrence of moulds and/or bacteria and biogases, which can harm human health by spreading in the air or result in undesirable/unpleasant odour. The disinfection system for this modification in installed in the environment to be disinfected, i.e. in the wastewater collection and recycling tank and its environment, which is near to gathering spots.

The disinfection system includes more than one ozone generator (18) suitable to provide ozone to wastewater (17), ozone detectors (6′) intended for measuring ozone concentration in the perimeter (3″) of the wastewater treatment tank (3′); at least one intermediate controller (11) for collecting and transferring data to the central controller (13); at least one another intermediate controller (12) for collecting and transferring data to the central controller (13) from identical disinfection systems; the central controller (13) for controlling ozone generators in order to generate ozone depending on the measured ozone concentration and the set time of the day; the timer (10); at least one presence detector (14); ozone-resistant ozone supply pipes (19); ozone release head (23) with outlets (21) and with reverse valves (22).

The ozone generators (18) are spreading ozone to the wastewater collection or recycling tank (3′) to be disinfected and are controlled through the central controller (13). Ozone produced by ozone generators (18) is directed straight to the wastewater (17). Ozone is released through ozone-resistant pipes (19), which are connected to the ozone release head (23) containing holes (21) with reverse valve (22) for releasing ozone, so that these holes would not be clogged with wastewater once ozone generators (18) stopped operating. Preferably, ozone generators (18) should be configured according to the size, air temperature and air humidity of the environment to be disinfected in order to increase efficiency of the generated ozone in dealing with microorganisms present in the very same environment. In this regard, efficiency of ozone as a disinfectant depends on several factors, including amount of used ozone, residual ozone content in the medium and other environmental factors, such as pH, temperature, amount of organic substances, etc.

The central controller (13) is intended to receive control information through the intermediate controller (11, 12), from the timer (10), the ozone detector (6′), and the presence detector (14), as well as the intermediate controllers (12) from other environments where identical disinfection systems are installed. The central controller (13) is intended for monitoring incoming control data and centrally adjusting settings by transmitting reciprocal directions through the intermediate controller (11, 12) for controlling ozone generator (18). Central controller (13) would allow to remotely monitor disinfection system in the wastewater pumping stations and control it. Central controller (13) comprises input for receiving response data from more than one intermediate controller (11, 12). Such way would allow observing and comparing data from several wastewater cleaning stations. Response data can be transferred from at least one intermediate controller (11, 12) to the central controller (13) by such means as cable, GSM module or internet connection.

The ozone detectors (6′) intended for measuring of ozone concentration in the air within the environment and in the protective perimeter, transfer captured data to the central controller (13). Ozone detectors (6′) are installed separately from the ozone generator (18) and mounted with a protective perimeter away from ozone generators (18), so that both devices would be separated as much as possible. This is to achieve that generated and not completely consumed zone, intended for wastewater disinfection and which could potentially be released to the surrounding air, would be mixed with the surrounding air before reaching the ozone detectors (6′). In this way, ozone detectors (6′) within the environment measure ozone concentration more accurately. When at least one ozone detector (6′) detects that ozone concentration in the monitored environment exceeds the set stopping value, the central controller (13) stops ozone generators (18). Thus excess ozone quantity generation is avoided. Preferably, that different concentration limits would be set depending on the time of the day. Thus, legal threshold values above which people would noticeably be affected by ozone would not be exceeded.

At least one presence detector (14) is installed in the wastewater treatment tank (3′) environment. Presence detector (14) sensor is directed to the place where movements of a person can be detected. Once a person is detected, the central controller (13) stops ozone generators (18). Thus a person could be safe in the environment of the wastewater treatment tank and avoid long-term impact of high ozone concentration that could impair his/her health. At least one motion detector can perform the function of presence detector (14). Various motion detectors can be used, such as infrared, microwave, ultrasound or tomography technologies. In addition, to increase presence detection accuracy, combined sensors can be used in the detectors.

The timer (10) is intended for controlling time of the day and control activities based on the time schedule programme as well as to transfer data to the central controller (13) through intermediate controller (11, 12) in order to control ozone generators (18) depending on the time of the day. Timer of this modification comprises a touch screen to enter the control options and to set the program time schedule. This is a simple way to control the timing of 24 hours according to the programmed schedule and set time of the day. When programming time schedule, one or more of the following components is taken into account: season, work days and holidays. Such way would allow for generated ozone vary within a day depending on a season and on a presence model. For example, at lower temperatures during winter, wastewater odour is less of a problem, compared to the summer months.

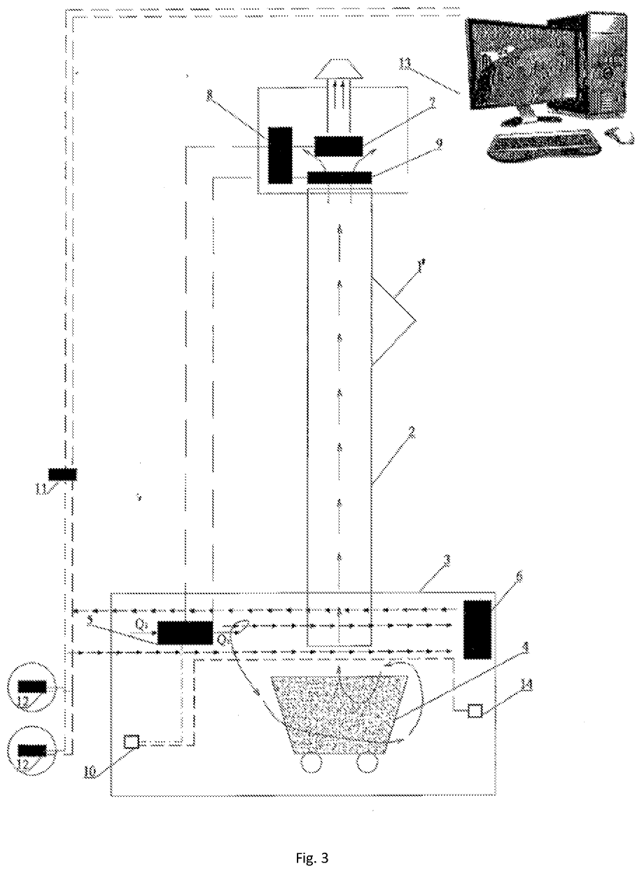

FIG. 3 shows disinfection system for disinfecting waste repositories with a waste chute pipe. The system comprises at least one ozone generator (5); the first ozone generator (6) intended for measuring ozone concentration in the waste repository room (3) and the second ozone detector (7) intended for measuring ozone concentration in the vents of waste repository (2); airflow detector (9); at least one intermediate controller (11) for collecting and transferring data to the central controller (13); at least one intermediate controller for other premises (12) for collecting and transferring data to the central controller (13) from identical disinfection systems; central controller (13) intended for controlling ozone generator, so that it would generate ozone depending on the measured ozone concentration and set time of the day; control relay (8); timer (10); at least one presence detector (14).

The disinfection system of this modification is installed in the waste repository (3), which is connected to the waste chute (2) that can be reached through collection door (1′). Ventilation chimney is at the top of the waste chute (2). When using waste collection system, a user throws trash through collection door (1′) and it falls down the chute (2) to the waste repository container (4), located in the waste repository.

The ozone generator (5) releases ozone to the waste repository (3) and is controlled by central controller (13), based on the data from the intermediate controller (11). Such data include timer (10) data, ozone detector (6, 7) data as well as presence detector (14) data. Ozone outlet of the ozone generator (5) is preferably adjusted to direct ozone gas flow to the waste container (4). Timer (10) should operate based on the time schedule programme and transfer data to the central controller (13) through the intermediate controller (11) in order to control ozone generator depending on the time of the day. Timer of this modification comprises a touch screen to enter the control options and to set the program time schedule.

In this modification, central controller (13) receives control information from the intermediate controller (11) and other intermediate controllers in the nearby identical waste collection rooms (12) that are connected to other disinfection systems located in other waste repositories, e.g., in the repositories of the same building or building complex. Central controller (13) monitors the incoming control data and centrally regulates settings by sending response instructions via the intermediate controller (11) to control the ozone generator. Once ozone concentration measured by ozone detectors (6, 7) fall below the permissible threshold, the central generator (13) can give a command to the ozone generator (5) to start generating more ozone. Thus formation of excessively high ozone concentration can be avoided. Central controller (13) comprises input to obtain response data from more than one intermediate controller from several waste storages. Such way would allow observing and comparing data from several waste storages. Response data can be transferred from the intermediate controller (11, 12) to the central controller by such means as cable, GSM module or internet connection.

All the detectors (6, 7, 14) transfer captured data to the intermediate controller (11), which in turn transmits the data to the central controller (13). Ozone detector (6) installed separately from the ozone generator (5) and is fixed to the wall of a waste repository (3), farther from the ozone generator and preferably on the opposite wall of the waste repository, so that both devices would be separated as much as possible. The aim is that generated ozone mixes up with room air before reaching the ozone detector (6). In this way, ozone sensor measures ozone concentration more accurately.

The presence detector (14) is installed on the walls/ceiling of the waste repository and is configured so that possibility of false alarms of such factors as falling waste or moving rodents would be reduced to a minimum. In this modification it is realized by directing the motion detector sensor to such a place only where movements of a person entering the waste storage can be detected. Of course, in addition, other technical solutions can be used, such as passive infrared detectors, set to ignore thermal footprints that are smaller than those of a human. In such cases only human presence is detected, while thermal radiation emitted by rodents is ignored. Presence detector (14) is used for detecting persons entering the waste repository (3): once it is activated, ozone generator is turned off (5). Although some ozone still remains the air of the waste repository (3), it is no longer actively generated; therefore a person can safely use the room. In addition, persons usually get to the waste repository through the entrance door and ozone is released outside once they are open. Thus ozone concentration in the waste repository is decreased even more and this happens once person entering the room is detected and ozone is no longer generated. In this way, ozone concentration decreases in the waste repository (3) when persons enter waste repository for, e.g. emptying the container (4) or cleaning the room manually. This prevents the impact of a high concentration of ozone, which may harm human health. Considering this aspect, speed at which ozone concentration lowers after opening the entrance door can also help deciding what concentration limit value can be set. For instance, such factors us doors opening outwards or too large waste repository entrance doors may result in dissipation of ozone from the waste repository. For this reason, higher ozone concentration threshold can be set in the central controller (13) in order to increase ozone concentration in the waste repository and thus achieve higher disinfection efficiency. Once persons enter the repository, ozone concentration drops to acceptable level, so that they would not be harmed. Central controller (13) can monitor the speed of ozone dissipation from the waste repository and respectively adjust the set limits of ozone concentration. Various presence detectors (14) can be used, such as infrared, microwave, ultrasound or tomography technologies. Other types of presence detectors also can be used, such as door sensors, which can detect when doors in waste repositories are opened. In addition, to increase presence detection accuracy, combined sensors can be used in the detectors.

The airflow detector (9) is installed at the top of the waste chute (2), by the junction with the ventilation chimney and is used for measuring the airflow released from the waste chute (2). Airflow detector (9) measures airflow released from the waste chute (2), obstruction of which results in decrease of the airflow going through the chute or in full suspension thereof. In such case, decrease of normal airflow can be detected by airflow detector (9) that reports to the central controller (13), which can deactivate the ozone generator (5) in order to avoid excessively high ozone concentration in the waste repository of a building.

The second ozone detector (7) measures ozone concentration in vent of a room (2). It can be installed to measure the ozone concentration level in the air that comes out of the flow detector (9). The control relay (8) is installed to transmit the obtained data back to the intermediate controller (11). Central controller stops the ozone generators (5, 18) when the second ozone sensor (7) detects that it exceeds the second concentration threshold value. It is the second safety measure that protects the repository room (3) and waste chute pipe (2) from formation of excessively high ozone concentration. Depending on the time of the day, it sets a different second threshold value. In this way, the system operates more actively during that time of the day, when it is less likely that repository holes shall be opened, thus resulting in higher ozone concentration.

The waste disinfection system monitors and adjusts amount of ozone in the air of waste repository as well as maintains balance between efficient disinfection and safe environment for the users of waste repository. For this purpose, when system is operated, timer (10) is used that sends data to the central controller (13) through intermediate controller (11) according to its time schedule programme in order to control ozone generator (5), so that it would generate varying quantity of ozone depending on the time; it can also be configured based on the amount of waste collected, air temperature, air humidity and waste disposal frequency in order to increase efficiency of generated ozone when dealing with the microorganisms present in that waste collection system. In this regard, efficiency of ozone as a disinfectant depends on several factors, including amount of used ozone, residual ozone content in the medium and other environmental factors, such as pH, temperature, relative humidity, and percentage of organic substances in the waste. For example, to obtain optimum efficiency, central controller (13) regulates activity of the ozone generator according to signal from the timer (10). For instance, it can activate ozone generator (5) during the night time, when waste repository (3) is usually not used, and to deactivate the generator (5) before the daytime comes, or ozone generator can remain activated, but the amount of released ozone is decreased. Also, it is possible to program the timer (10) according to the season, workdays and holidays. Hence, the timer (10) provides waste repository users with a possibility for flexible adjustment of ozone concentration thresholds according to a number of environmental factor, personal settings and waste repository contamination level by not exceeding the legal limitations at the same time. For this purpose, ozone detectors (6, 7) are used to detect when ozone concentration exceeds the set limits in both waste repository (3) and waste chute (2) respectively and then central controller (13) deactivates ozone generator (5) in order to prevent ozone concentration from increasing. The set values within the places above may vary, as waste repository can (3) accept higher ozone concentration than that in the waste chute (2), where, in case of its high concentration, ozone can flow through holes to residential floors of the building. Also, by using time schedule, it is possible to set different thresholds depending on the time of the day. For example, at the night time, high ozone concentration limit value is set according to the maximum permissible limit values provided by law, and, on the contrary, at the day time, when it is more likely that the waste storage facility will be used by people, it is possible to set a lower ozone concentration limit, which would allow for the building residents not to feel the effect of ozone. It is a simple way to control a 24-hour schedule, although other ways are also possible. For instance, outdoor light and/or temperature detectors can be installed in order to distinguish day and night time.

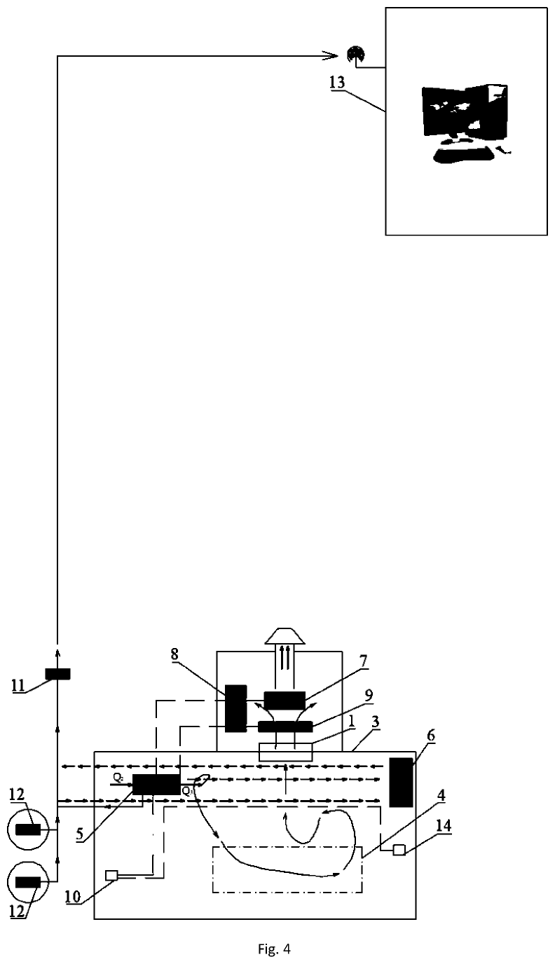

FIG. 4 shows disinfection system for installation in premises where certain conditions can cause appearance of fungus and/or bacteria that may pose risk to human health by spreading in the air. Or which result in at least unwanted/unpleasant odour. Such premises may be intended for food and non-food materials' storage, changing clothes in gyms, etc. Disinfection system of such modification is installed in the room to be disinfected (3), which is connected to the vent opening (1). The system comprises at least one ozone generator (5); the first (6) ozone detector intended for measuring ozone concentration in the wastewater room (3) and the second ozone detector (7) intended for measuring ozone concentration in the wastewater pumping stations, located in the vents of the system (1); airflow detector (9); at least one intermediate controller (11) intended for collecting and transferring data to the central controller (13); at least one intermediate controller for other premises (12), intended for collecting and transferring data to the central controller (13) from other identical disinfection systems; central controller (13) intended for managing ozone generators, so that it would generate ozone depending on the measured ozone concentration and set time of the day; control relay (8); timer (10); at least one presence detector (14).

Premises to be disinfected (3) comprise any rooms to be disinfected accessible to a person, or which are near the crowded spaces, and where at certain conditions fungus and/or bacteria may appear, which may be harmful to human health. Or which result in at least unwanted/unpleasant odour. Examples of such premises are storages of vending machines for collecting empties (packaging waste), where such vending machines are installed to transfer empties (packaging waste) into the waste storage space. Also, such premises as warehouses for food and non-food materials, container trucks/refrigerators, changing rooms in gyms and any other similar premises or facilities as mentioned earlier.

The ozone generator (5) spreads the ozone to the room to be disinfected (3) and is controlled through the central controller (13). Ozone release outlet of the ozone generator (5) is adapted for directing ozone gas flow to the room intended for the room to be disinfected and/or facility (4) to be disinfected within the room, such as automated tare containers, waste recycling equipment, food and non-food processing equipment, etc.

In this modification, the central controller (13) obtains control information from the timer (10), ozone detectors (6, 7) and presence detector (14) through intermediate controller (11), as well as other identical intermediate controllers (12) intended for disinfection systems in nearby rooms. Central controller (13) monitors the incoming control data and centrally regulates settings by sending response instructions via the intermediate controller (11) to control the ozone generator. Central controller (13) comprises input to obtain response data from more than one intermediate controller from several waste storages. Such way would allow observing and comparing data from several waste storages. Response data can be transferred from the intermediate controller (11, 12) to the central controller by such means as cable, GSM module or internet connection.

The room to be disinfected (3) contains the first ozone detector (6), intended for measuring ozone concentration inside the room (3). The first ozone detector (6) is installed separately from the ozone generator (5); it is fitted to the wall in premises (3), away from the ozone generator, and preferably on the opposite wall in premises (3), to make both devices separated as much as possible. The aim is that generated ozone mixes up with room air before reaching the ozone detector (6). Thus, the ozone detector (6) installed in the room measures ozone concentration more accurately.

The presence detector (14) is installed on the walls/ceiling of the waste repository and is configured so that possibility of false alarms of such factors as falling waste or moving rodents would be reduced to a minimum. In this modification it is realized by directing the motion detector sensor to such a place only where movements of a person entering the waste storage can be detected. Of course, in addition, other technical solutions can be used, such as passive infrared detectors, set to ignore thermal footprints that are smaller than those of a human. In such cases only human presence is detected, while thermal radiation emitted by rodents is ignored. Presence detector (14) is used for detecting persons entering the room to be disinfected (3): once it is activated, ozone generator is turned off (5). Although there is still ozone remaining the air of the room to be disinfected (3), it is no longer actively generated; therefore, a person can safely use the room. In addition, persons usually get to the room to be disinfected through the entrance door and ozone is released outside once they are open. Thus ozone concentration in the room to be disinfected is decreased even more and this happens once person entering the room is detected and ozone is no longer generated. This prevents the impact of a high concentration of ozone, which may harm human health. Considering this aspect, speed at which ozone concentration lowers after opening the entrance door can also help deciding what concentration limit value can be set. For this reason, higher ozone concentration threshold can be set in the central controller (13) in order to increase ozone concentration in the room to be disinfected and thus achieve higher disinfection efficiency. Once persons enter the room to be disinfected, ozone concentration drops to acceptable level, so that they would not be harmed. Central controller (13) can monitor the speed of ozone dissipation from the room to be disinfected and respectively adjust the set limits of ozone concentration. Various presence detectors (14) can be used, such as infrared, microwave, ultrasound or tomography technologies. Other types of presence detectors also can be used, such as door sensors, which detect when doors in waste repositories are opened. In addition, to increase presence detection accuracy, combined sensors can be used in the detector (14).

The airflow detector (9) measures airflow released from the vent opening (1) Due to malfunction of the ventilation openings (1), air flow through the ventilation openings is reduced or substantially lost. In such case, decrease of normal airflow can be detected by airflow detector (9) that reports about it to the central controller (13) via the intermediate controller (11). Then the central controller (13) stops the ozone generator (5) in order to avoid excessively high ozone concentration in the room to be disinfected.

The second ozone detector (7) measures ozone concentration in vent of a room (1). It can be installed to measure the ozone concentration level in the air that comes out of the flow detector (9). The control relay (8) is installed to transmit the obtained data back to the intermediate controller (11). Central controller stops the ozone generator (5) when the second ozone sensor (7) detects that it exceeds the second concentration threshold value. It is the second safety measure that protects the room to be disinfected (3) and vent opening (1) from formation of excessively high ozone concentration. Depending on the time of the day, it sets a different second threshold value. In this way, the system operates more actively during that time of the day, when it is less likely that openings of the room to be disinfected shall be opened, thus resulting in higher ozone concentration.

The disinfection system monitors and regulates the amount of ozone in the air of the room to be disinfected and maintains a balance between effective disinfection and safe environment by using a timer (10), which is programmed according to time schedule programme and sends data to the central controller (13) through intermediate controller (11) in order to control ozone generator (5), so that it would generate varying quantity of ozone depending on the time; it can also be configured based on the size of the room to be disinfected, air temperature, and air humidity. In this regard, efficiency of ozone as a disinfectant depends on several factors, including amount of used ozone, residual ozone content in the medium and other environmental factors, such as pH, temperature, relative humidity, and percentage of organic substances in the waste. For example, to obtain optimum efficiency, central controller (13) regulates activity of the ozone generator according to signal from the timer (10). For instance, it can activate ozone generator (5) during the night time, when room to be disinfected (3) is usually not used, and to deactivate the generator (5) before the daytime comes, or ozone generator can remain activated, but the amount of released ozone is decreased. Also, it is possible to program the timer (10) according to the season, workdays and holidays. Hence, the timer (10) provides the users of the room to be disinfected with a possibility for flexible adjustment of ozone concentration thresholds according to a number of environmental factor, personal settings and contamination level by not exceeding the legal limitations at the same time. For this purpose, ozone detectors (6, 7) are used to detect when ozone concentration exceeds the set limits in both premises to be disinfected (3) and vent opening (1) respectively and then central controller (13) deactivates ozone generator (5) in order to prevent ozone concentration from increasing. The set values for the places above may vary, as for the rooms to be disinfected (3) higher ozone concentrations can be acceptable than that in the vent openings (1). Also, by using time schedule, it is possible to set different thresholds depending on the time of the day. For example, at the night time, high ozone concentration limit value is set according to the maximum permissible limit values provided by law, and, on the contrary, at the day time, when it is more likely that the room to be disinfected will be used by people, it is possible to set a lower ozone concentration limit. It is a simple way to control a 24-hour schedule, although other ways are also possible. For instance, outdoor light and/or temperature detectors can be installed in order to distinguish day and night time.

Each individual disinfection system can operate as a standalone system, but such a system cannot be immune from mistakes and there may be false alarms due to, for example, power supply failures, user errors, poor technical condition, surges, lightning or equipment failures, so connection to a central controller (13) provides the capability to remotely monitor individual disinfection systems. Such a configuration allows monitoring disinfection systems around the clock and quickly reacting in cases of network faults, for example, when ozone concentration exceeds the legal limits.

Although numerous characteristics and advantages together with structural details and features have been listed in the present description of invention, the description is provided as an example fulfilment of the invention. Without departing from the principles of the invention, there may be changes in the details, especially in the form, size and layout, in accordance with most widely understood meanings of the concepts and definitions used in claims. It should be understood that design of the disinfection system can be changed at least in such a way that several ozone generators and/or ozone sensors are included, which can be installed in various places of the premises or environment to be disinfected depending on the parameters of the premises or environment to be disinfected, in order to ensure maximum disinfection efficiency within the object that is framed by the present invention.

1. A disinfection system comprising an ozone generator, comprising:
at least one ozone generator (5, 18) for generating ozone in gas and/or liquid media;
a first ozone detector (6, 6′) to measure the concentration of ozone in the gaseous medium;
a programmable timer (10);
a presence detector (14);
at least one main system process management tool (13);
at least one intermediate system process management tool (11);

2. The disinfection system according to claim 1, wherein the timer (10) is programmed according to a schedule and set time of a day.

3. The disinfection system according to claim 2, wherein said programmed schedule includes data on time of a year, workdays and holidays.

4. The disinfection system according to claim 1, wherein the main system process management tool (13) is a central controller designed to control the at least one ozone generator (5, 18) according to a set ozone level.

5. The disinfection system according to claim 1, wherein said central controller (13) is designed to stop the at least one ozone generator (5, 18) based on stop value for the time of the day.

6. The disinfection system according to claim 1, wherein the first ozone detector (6, 6′) is installed separately from the ozone generators (5, 18).

7. The disinfection system according to claim 1, wherein the central controller (13) is designed to stop the ozone generators (5, 18) if the presence detector (14) detects the presence of a person nearby the ozone generators (5, 18).

8. The disinfection system according to claim 7,
wherein the presence detector (14) is a motion detector.

9. The disinfection system according to claim 1, further comprising an air flow detector (9), designed to measure air flow going out of the room to be disinfected (3) inside an air vent for the room being disinfected, and
wherein the central controller (13) is designed to stop the ozone generators (5, 18) if the air flow detector (9) detects that the air flow speed has dropped below the threshold value.

10. The disinfection system according to claim 1, further comprising a second ozone generator (7), designed to measure ozone levels inside the air vent (1, 2) through which the air goes out of the room being disinfected and
wherein the central controller (13) is designed to stop the ozone generators (5, 18) if the second ozone detector (7) detects that ozone level exceeds the set second stop value.

11. The disinfection system according to claim 10,
wherein the central controller (13) is configured to have different second stop values depending on the time of the day.

12. The disinfection system according to claim 1, wherein the intermediate controller (11) is connected to the central controller (13) for data exchange purposes.

13. The disinfection system according to claim 1, wherein the central controller (13) includes an input for receiving feedback data from more than one intermediate controller (11, 12) in several rooms to be disinfected.

14. The disinfection system according to claim 12, wherein the feedback data is transmitted from the intermediate controller (11) to the central controller (13) via a cable, a GSM module or an internet connection.

15. A method to control ozone level in a disinfected environment, comprising control of ozone generators, wherein the ozone level in at least one medium in environment for disinfecting is controlled via the central controller (13) according to the at least one ozone sensor (6, 7) and the first and/or second ozone level values, where the first and second ozone level values are assigned to remote areas; and/or the signal of presence detector (14); and/or timer (10) data, where the timer data includes data of time of the day and/or time of the year.

16. The disinfection system according to claim 2, wherein the main system process management tool (13) is a central controller designed to control the at least one ozone generator (5, 18) according to a set ozone level.

17. The disinfection system according to claim 3, wherein the main system process management tool (13) is a central controller designed to control the at least one ozone generator (5, 18) according to a set ozone level.

18. The disinfection system according to claim 2, wherein said central controller (13) is designed to stop the at least one ozone generator (5, 18) based on stop value for the time of the day.

19. The disinfection system according to claim 3, wherein said central controller (13) is designed to stop the at least one ozone generator (5, 18) based on stop value for the time of the day.

20. The disinfection system according to claim 4, wherein said central controller (13) is designed to stop the at least one ozone generator (5, 18) based on stop value for the time of the day.
